(19) United States
(12) Patent Application Publication
(10) Pub. No.: US 2018/0299360 A1
(43) Pub. Date: Oct. 18, 2018

(54) METHODS FOR SELECTIVELY ANALYZING BIOLOGICAL SAMPLES

(71) Applicant: Seoul National University R&DB Foundation, Seoul (KR)

(72) Inventors: Sung Hoon KWON, Seoul (KR); Yun Jin Jeong, Seoul (KR); Sung Silk Kim, Seoul (KR); Jin Hyun Kim, Seoul (KR)

(21) Appl. No.: 15/958,566

(22) Filed: Apr. 20, 2018

Related U.S. Application Data
(63) Continuation-in-part of application No. PCT/KR2016/011941, filed on Oct. 21, 2016.

(30) Foreign Application Priority Data
Oct. 21, 2015 (KR) 10-2015-0146984

(51) Int. Cl. G01N 1/44; C12Q 1/6874; C12Q 1/6848

(52) U.S. Cl. CPC G01N 1/44; C12Q 1/6848; C12Q 1/6874

(57) ABSTRACT

Provided is a method for selectively analyzing biological samples. The method includes: preparing a substrate on which biological samples are arranged; dividing the substrate into areas where one or more target specimens are located and areas where one or more non-target specimens are located; forming a masking structure to selectively mask the areas where the non-target specimens are located; introducing a biochemical reaction reagent into the areas where the target specimens are located, such that the biochemical reaction reagent reacts with the target specimens; and analyzing the reacted target specimens on the substrate or recovering the reacted target specimens from the substrate and analyzing the recovered target specimens.

### CROSS-REFERENCE TO RELATED APPLICATIONS

This application is a Continuation-In-Part of Application No. PCT/KR2016/011941, filed Oct. 21, 2016 which in turn claims the benefit of Korean Patent Application No. 10-2015-0146984, filed Oct. 21, 2015, the disclosures of which are incorporated by reference into the present application.

### TECHNICAL FIELD

The present disclosure relates to methods for selectively analyzing biological samples, and more specifically to methods for sorting biological samples with high accuracy while maintaining their original morphology and analyzing the sorted biological samples.

### BACKGROUND ART

A tissue is an example of biological sample and consists of a vast number of cells, all of which do not have the same DNA or RNA. Thus, there is a need to develop a technique for sorting only cancer cells in high purity to accurately determine whether cancer tissues have certain mutations or abnormalities.

For example, U.S. Patent Publication No. 2014-0093911, PCT Publication No. WO2013-130714, and U.S. Patent Publication No. 2014-0357511 disclose methods for sorting and analyzing biological samples.

In comparison with techniques for sorting samples based on surface physical properties of materials, techniques for sorting samples based on the size or surface fluorescence of materials enable precise sorting of samples but have disadvantages in that samples lose their original tissue morphology or arrangement during elution, the sorting criteria are limited to a few characteristics, causing low specificity, and no image information is considered. Techniques for sorting samples through image information on cells placed in microwells after elution are also disadvantageous in that the original morphology is not preserved in a natural state.

As described above, it is generally known that samples are sorted after elution with solutions for convenience of handling. However, the characteristics of target specimens are determined by not only their own images but also their surrounding images. It is also difficult to accept that the eluted samples in the form of solutions are the same as their original morphology. Thus, there is a need to develop a technique for sorting samples that are coated without changing their original morphology.

### DETAILED DESCRIPTION OF THE INVENTION

### Means for Solving the Problems

One aspect of the present disclosure provides a method for selective analysis of biological samples, comprising the steps of: preparing a substrate on which biological samples are arranged; dividing the substrate into areas where one or more target specimens are located and areas where one or more non-target specimens are located; forming a masking structure to selectively mask the areas where the non-target specimens are located; introducing a biochemical reaction reagent into the areas where the target specimens are located, such that the biochemical reaction reagent reacts with the target specimens; and analyzing the reacted target specimens on the substrate or recovering the reacted target specimens from the substrate and analyzing the re target specimens.

A further aspect of the present disclosure provides a method for selective analysis of biological samples, comprising the steps of: preparing a substrate on which biological samples are arranged; dividing the substrate into areas where one or more target specimens are located and areas where one or more non-target specimens are located; forming a masking film layer on the substrate to selectively mask the areas where the non-target specimens are located; peeling the masking film layer from the substrate to remove the non-target specimens, leaving the target specimens on the substrate; introducing a biochemical reaction reagent into the areas where the target specimens are located, such that the biochemical reaction reagent reacts with the target specimens or recovering the target specimens from the substrate and reacting a biochemical reaction reagent with the recovered target specimens; and analyzing the reacted target specimens.

Another aspect of the present disclosure provides a method for selective analysis of biological samples, comprising the steps of: preparing a substrate on which biological samples are arranged; dividing the substrate into areas where one or more target specimens are located and areas where one or more non-target specimens are located; forming a masking structure to selectively mask the areas where the non-target specimens are located; introducing a lysis solution into the areas where the target specimens are located, to lyse the target specimens; reacting nucleic acid molecules originating from the target specimens by the lysis with a biochemical reaction reagent to prepare libraries of the nucleic acid molecules for sequencing; recovering the libraries from the substrate; and sequencing the recovered libraries.

Another aspect of the present disclosure provides a method for selective treatment of biological samples, comprising the steps of: preparing a substrate on which biological samples are arranged; dividing the substrate into areas where one or more target specimens are located and areas where one or more non-target specimens are located; forming a masking structure to selectively mask the areas where the non-target specimens are located; and introducing a biochemical reaction reagent into the areas where the target specimens are located, such that the biochemical reaction reagent reacts with the target specimens.

Another aspect of the present disclosure provides a method for selective treatment of biological samples, comprising the steps of: preparing a substrate on which biological samples are arranged; dividing the substrate into areas where one or more target specimens are located and areas where one or more non-target specimens are located; forming a masking structure to selectively mask the areas where the non-target specimens are located; peeling the masking structure together with the masked non-target specimens from the substrate to remove the non-target specimens, leaving the target specimens on the substrate; and introducing a biochemical reaction reagent into the areas where the target specimens are located or recovering the target specimens from the substrate and reacting a biochemical reaction reagent with the recovered target specimens.

Another aspect of the present disclosure provides a method for selective treatment of biological samples, comprising the steps of: preparing a substrate on which biological samples are arranged; dividing the substrate into areas where one or more target specimens are located and areas where one or more non-target specimens are located; selectively bonding or lysing the areas where the target specimens are located, to selectively extract constituents of the target specimens; and recovering the target specimens from the substrate and reacting a biochemical reaction reagent with the recovered target specimens.

Another aspect of the present disclosure provides a method for selective analysis of biological samples, comprising the steps of: preparing a substrate on which biological samples are arranged; dividing the substrate into areas where one or more target specimens are located and areas where one or more non-target specimens are located; providing a first microfluidic structure on the substrate; introducing a curable material into the first microfluidic structure; selectively applying energy to the curable material present in the areas where the non-target specimens are located, such that the curable material is cured to form a masking structure; introducing a biochemical reaction reagent into the areas where the target specimens are located, such that the biochemical reaction reagent reacts with the target specimens; and analyzing the reacted target specimens on the substrate or recovering the reacted target specimens from the substrate and analyzing the recovered target specimens.

Another aspect of the present disclosure provides an apparatus for selective treatment of biological samples, comprising: a unit for providing a first microfluidic structure forming a masking structure on a substrate on which biological samples are arranged; a unit for introducing a curable material into the first microfluidic structure; a unit for forming a masking structure by applying energy to masking areas as per a user's request or a predetermined algorithm to cure the masking areas; a unit for removing the first microfluidic structure from the substrate; a unit for providing a second microfluidic structure adapted to retain a biochemical reaction reagent on the substrate; and a unit for biochemical treatment by introducing a biochemical reaction reagent into separate spaces between the second microfluidic structure and the substrate or applying energy of light, heat, agitation, vibration or sound waves to the separate spaces such that a biochemical reaction takes place.

Another aspect of the present disclosure provides an apparatus for selective treatment of biological samples, comprising: a unit for providing a first microfluidic structure forming a masking structure on a substrate on which biological samples are arranged; a unit for introducing a curable material into the first microfluidic structure; a unit for forming a masking structure by applying energy to masking areas as per a user's request or a predetermined algorithm to cure the masking areas; and a unit for biochemical treatment by introducing a biochemical reaction reagent into the first microfluidic structure or applying energy of light, heat, agitation, vibration or sound waves to the first microfluidic structure such that a biochemical reaction takes place.

Yet another aspect of the present disclosure provides an apparatus for selective treatment of biological samples, comprising: a unit for introducing a curable material into a first microfluidic structure; a unit for forming a masking structure by applying energy to masking areas as per a user's request or a predetermined algorithm to cure the masking areas; and a unit for biochemical treatment by introducing a biochemical reaction reagent into the first microfluidic structure or applying energy of light, heat, agitation, vibration or sound waves to the first microfluidic structure such that a biochemical reaction takes place, wherein the first microfluidic structure is provided on a substrate on which biological samples are arranged.

### Effects of the Invention

The present disclosure enables the separation of biological samples with high specificity based on their kinds and locations. In addition, the present disclosure enables the analysis of target specimens while maintaining their original structure and morphology in areas where the target specimens are located because the structures are prepared while maintaining their coated state. Furthermore, according to the present disclosure, there is no need to transfer target specimens to a corresponding container or substrate for a subsequent reaction, reducing the probability of contamination and ensuring high accuracy. Moreover, according to the present disclosure, an existing biochemical methodology can be applied to separated target specimens without any additional system.

### BRIEF DESCRIPTION OF THE DRAWINGS

FIG. 1 is a flow chart illustrating a method for selective analysis of biological samples according to one embodiment of the present disclosure.

FIG. 2 illustrates two methods for selective analysis of biological samples according to exemplary embodiments of the present disclosure: specifically, 3-1 and 4 of FIG. 2 illustrate a method based on a reaction of target specimens with a biochemical reaction reagent in a state in which a masking structure remains unremoved on a substrate, and 3-2 of FIG. 2 illustrate a method based on a reaction of target specimens with a biochemical reaction reagent after removal of a masking structure from a substrate.

FIG. 3 diagrammatically illustrates the embodiment of FIG. 1.

FIG. 4 illustrates a procedure for forming a masking structure using a microfluidic structure according to one embodiment of the present disclosure.

FIG. 5 illustrates procedures for supplying and recovering a biochemical reaction reagent using a microfluidic structure according to exemplary embodiments of the present disclosure.

FIG. 6 indicates that the inventors can peel non-target samples in order to leave only target samples on the substrate or that the inventors can selectively fix target samples on the film layer.

FIG. 7 indicates that the inventors don't have to generate film layer on the substrate with samples. The inventors can prepare another substrate to have adhesive structures which can be fit to the target or non-target samples after alignment. Then, the inventors can align and attach the substrate with samples and the substrate with adhesive structures. The inventors then can perform biological assay to the sorted target samples.

FIG. 8 indicates that the inventors can prepare another substrate with adhesive structure in many ways. The inventors can fabricate innately adhesive structure on another structure or can cover adhesive after fabricating structure on another substrate.

FIG. 9 illustrates an apparatus for selective treatment of biological samples according to one embodiment of the present disclosure.

### MODE FOR CARRYING OUT THE INVENTION

Embodiments of the present disclosure will now be described in detail with reference to the accompanying drawings. These embodiments are provided so that this disclosure will be thorough and complete, and will fully convey the scope of the present disclosure to those skilled in the art. Accordingly, the present disclosure may be embodied in many different forms and should not be construed as limited to the exemplary embodiments set forth herein. In the drawings, the dimensions, such as widths, lengths and thicknesses, of elements may be exaggerated for clarity. The drawings are explained from an observer's point of view. It will be understood that when an element is referred to as being “on” another element, it can be directly on the other element, or one or more intervening elements may also be present therebetween.

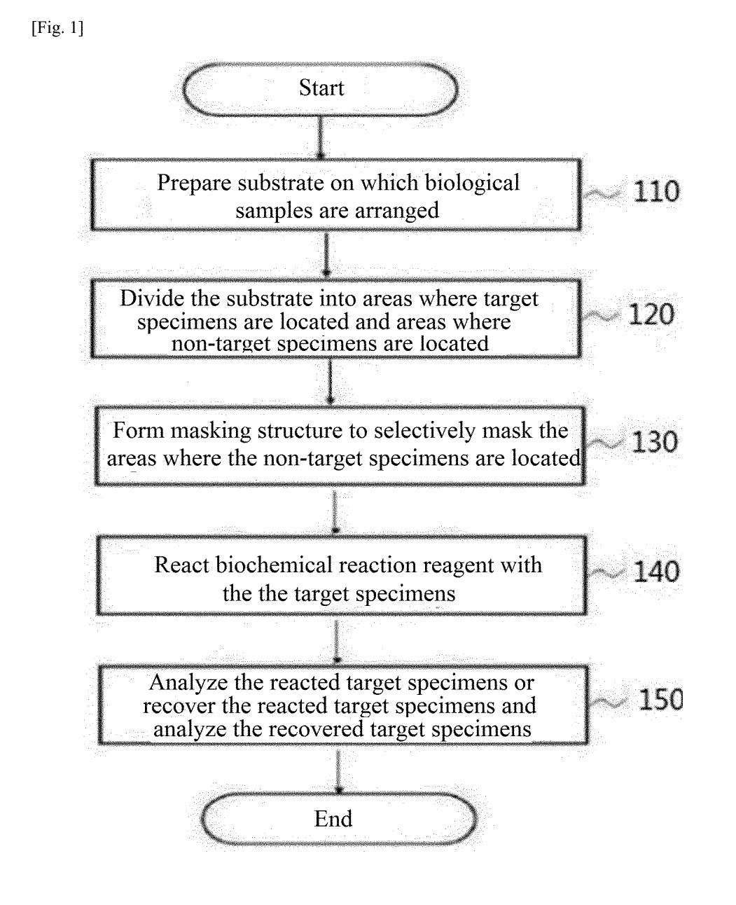

FIG. 1 is a flow chart illustrating a method for selective analysis of biological samples according to one embodiment of the present disclosure. Referring to FIG. 1, a substrate on which biological samples are arranged is prepared (step 110). The biological samples include target specimens and non-target specimens.

The biological samples may be selected from the group consisting of tissues, blood, cells, DNAs, RNAs, proteins, exosomes, metabolites, biopsy specimens, and mixtures thereof.

The biological samples may be provided on the substrate by suitable techniques, such as stamping, rolling, smearing, capillary action, microfluidics, and pipetting dispensing.

Any substrate that provides a surface for supporting the biological samples may be used without particular limitation. The substrate may be selected from the group consisting of slide glass, microbeads, nanoparticles, nanostructures, capillaries, microfluidic supports, porous structures, spongy structures, dendrimers, and combinations thereof. The substrate may be one whose surface is partially or fully functionalized with one or more chemical functional groups or one or more substances selected from DNAs, RNAs, and proteins. The substrate may be made of glass, silicon or a polymeric material. For example, the substrate may be slide glass. The substrate may be a functionalized substrate modified with one or more substances selected from the group consisting of DNAs, RNAs, proteins, antibodies, and chemicals. For example, the substrate may be a microarray substrate integrated with biological samples such as DNAs and proteins or a massively parallel sequencing substrate.

In step 120, the kinds and locations of the biological samples are read to divide the substrate into areas where the target specimens are located and areas where the non-target specimens are located.

The kinds and locations of the biological samples may be read in various ways, for example, by image observation, fluorescence signals or coordinate information. Staining of the biological samples may provide image information. Any staining technique that can provide information on the biological samples may be used without limitation. For example, the staining technique may be selected from the group consisting of Giemsa staining, hematoxylin and eosin (H&E) staining, fluorescence in situ hybridization (FISH) staining, immunofluorescence (IF) staining, and immunohistochemistry (IHC) staining. The image observation may be performed using a suitable tool such as an optical microscope or electron microscope. In some embodiments, the target specimens may be sorted by direct observation with naked eyes through an optical microscope or electron microscope or in an automated fashion using a separate software to obtain positional information of the biological samples. The substrate may be a DNA microarray substrate. In this case, several spots may be sorted based on their known coordinate information although they are not visible by imaging. For example, after imaging of the biological samples, the target specimens may be divided into several or several tens of groups using a clustering or classification technique in an automated fashion, followed by automatic or manual sorting.

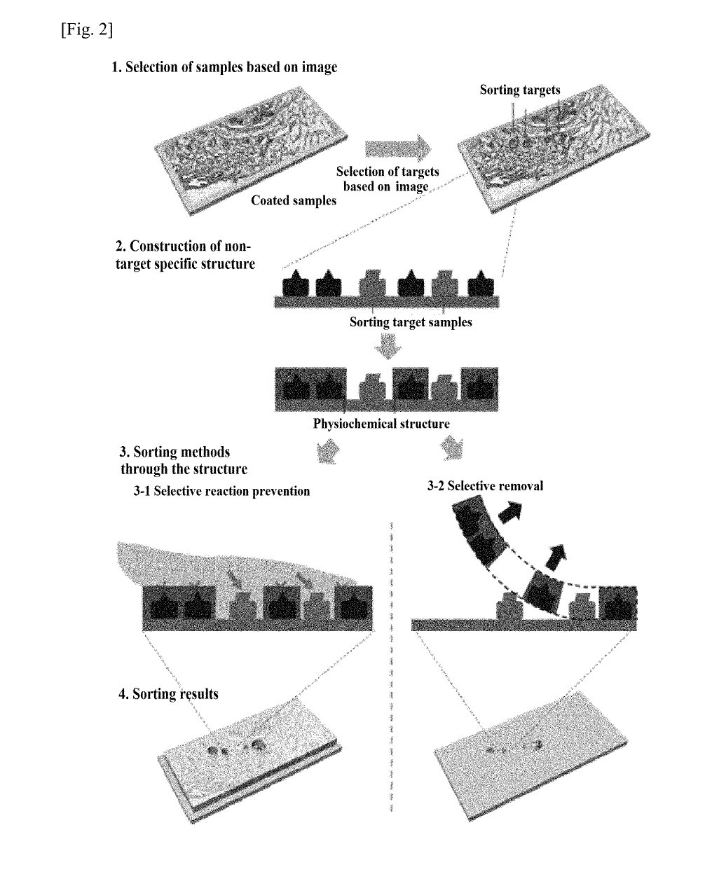

FIG. 2 illustrates two methods for selective analysis of biological samples according to exemplary embodiments of the present disclosure. Specifically, 3-1 and 4 of FIG. 2 illustrate a method based on a reaction of target specimens with a biochemical reaction reagent in a state in which a masking structure remains unremoved on a substrate, and 3-2 of FIG. 2 illustrate a method based on a reaction of target specimens with a biochemical reaction reagent after removal of a masking structure from a substrate.

Referring to 1 of FIG. 2, the kinds and locations of biological samples arranged on a substrate are read by image observation, fluorescence signals or coordinate information to divide the substrate into areas where sorting targets are located and areas where non-target specimens are located.

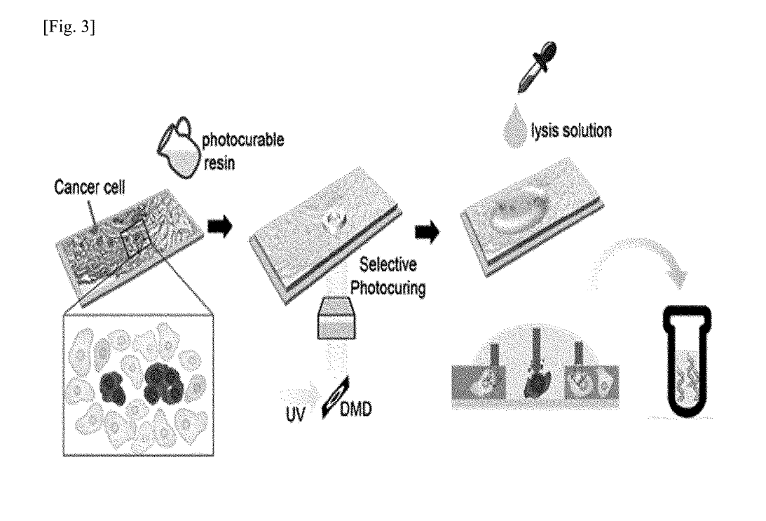

FIG. 3 diagrammatically illustrates the embodiment of FIG. 1. As can be seen from the left diagram of FIG. 3, targets suspected as cancer cells are sorted during image observation of biological samples.

According to the present disclosure, the kinds and locations of the biological samples can be accurately distinguished by image observation, fluorescence signals or coordinate information. Accordingly, the samples can be sorted while maintaining their coated state.

In step 130, a masking structure is formed to selectively mask the areas where the non-target specimens are located.

In one embodiment, a masking structure may be constructed by coating a liquid masking material over the entire surface of the substrate on which the biological samples are mounted and selectively applying a physiochemical action on the selected areas.

Referring to 2 of FIG. 2, a masking structure is formed to selectively mask the areas where the non-target specimens distinguished by reading the kinds and locations of the biological samples are located. That is, the target specimens of interest are exposed for a subsequent biochemical reaction and some or all of the non-target specimens are masked with the predetermined structure to prevent a biochemical reaction reagent from infiltrating into the surrounding non-target specimens.

The masking structure may be made of a material physically or chemically protected against the attack of a biochemical reaction reagent. For example, the masking structure may be made of at least one material selected from the group consisting of polymer resins, waxes, metals, metal oxides, and glass.

In one embodiment, the formation of the masking structure may include coating a curable material on the substrate and curing the curable material by light or heat. The curable material may include an unsaturated monomer. Non-limiting examples of such unsaturated monomers include ethoxylated trimethylolpropane triacrylate, curable epoxy (available under the trade name NOA), polyethylene glycol diacrylate, polypropylene glycol diacrylate, and polyurethane acrylate. These unsaturated monomers may be used alone or in combination. For example, polyethylene diacrylate may be crosslinked into a three-dimensional structure by free-radical polymerization due to the presence of acrylate groups at both ends of the polyethylene glycol chain. The curable material may be any type of liquid medium that can be converted to solid.

The curable material may further include a nanomaterial that converts electromagnetic waves into heat or an or initiator that induces free-radical polymerization by an external energy source. The initiator may be an azo-based compound or a peroxide. The curable material may further include a proper crosslinking agent. Examples of such cross-linking agents include N,N′-methylenebisacrylamide, methylenebismethacrylamide, and ethylene glycol dimethacrylate. Suitable energy sources for curing may include heat, UV light, visible right, infrared light, and electron beam.

The curable material may be bonded to the substrate when cured. For example, the curable material forms a bond with glass during curing so that the masking structure can be more strongly fixed to the substrate.

The curable material may be infiltrated into and cured in the biological samples. For example, when mixed with an alkaline lysis reagent or a proteinase, the curable material may be infiltrated between and into tissues and cured by an external energy source.

The biological samples may be pretreated before supply of the curable material for better infiltration of the curable material. For example, the biological samples may be subjected to a chemical reaction to form pores in the cell membranes before supply of the curable material. Alternatively, the biological samples may be subjected to a biochemical reaction to lyse the cell membranes without lysis of the nuclear membranes before supply of the curable material. As a result of this biochemical reaction, only nuclei are left in the biological samples.

In one embodiment, the formation of the masking structure may include coating the curable material over the entire surface of the substrate, curing the curable material by light or heat, and removing the uncured curable material. In another embodiment, the formation of the masking structure may include dispensing the curable material along the shape of the masking structure and curing the curable material by light or heat. In another embodiment, the formation of the masking structure may include supplying the curable material through a microfluidic chip or chamber.

Various patterning techniques may be utilized for selectively masking the non-target specimens. For example, the masking structure may be formed by at least one technique selected from the group consisting of lithography, laser scanning, inkjet printing, and 3D printing. Specifically, the masking structure may be formed by a general mask lithography process or a maskless lithography process using a digital mirror device (DMD).

In this connection, the curable material is coated on the areas where the non-target specimens are located and optofluidic maskless lithography is performed to construct the masking structure at the corresponding locations, as illustrated in the middle diagram of FIG. 3.

After sorting of the target specimens, the curable material may be coated over the entire surface of the substrate to form the masking structure on a large area. Alternatively, the curable material may be coated on the areas of the non-target specimens located in the vicinity of the target specimens rather than the areas of the non-target specimens distant from the desired target specimens. In this case, the masking structure is formed only in necessary portions.

Lithography for the formation of the masking structure may be performed over a large area without using a lens between a mask and the substrate. Alternatively, lithography may be sequentially performed on several areas of the curable material with high resolution using a lens. Particularly, lithography using a lens enables sequential formation of masking structures through one or several types of stationary masks even when the biological samples are substituted with different biological samples, and as a result, the target areas are changed, avoiding the need to replace the masks whenever the samples are changed.

The sequential lithography on the biological samples using a lens may further an optimized algorithm for forming the masking structure on the desired areas of the target specimens using one or several types of stationary masks. Alternatively, the sequential lithography may further an optimized algorithm for forming the masking structure on the desired areas of the target specimens using a digital mirror device (DMD).

The lithography using one or several types of stationary masks may further include controlling the size and resolution of the masking structure by varying the magnification of the lens.

The masking structure may be patterned by photolithography using a patterned mask. For example, the pattern may be a grid masking pattern. Photolithography enables the formation of a patterned masking structure accommodating the target specimens instead of forming a masking structure only in the vicinity of the sorted target specimens. A masking structure can be formed in a simple manner by photolithography compared to by maskless lithography.

The masking structure may be formed by the supply of a hydrophilic or hydrophobic coating agent. Preferably, a hydrophobic coating agent is supplied to the periphery of the target specimens to form the masking structure and an aqueous solution is supplied to the interior of the masking structure.

The masking structure may be previously formed and mounted on or assembled to the substrate.

Since the formation of the masking structure does not adversely affect the biological samples, the biological samples can be cultured after formation of the masking structure. For example, after the masking structure is selectively formed around cells with a desired phenotype, the cells may be cultured separately from cells with other phenotypes.

In step 140, a biochemical reaction reagent is introduced into the areas where the target specimens are located and is allowed to react with the target specimens.

For example, the biochemical reaction reagent may be selected from the group consisting of lysis solutions, PCR reagents, reagents for whole genome amplification, reagents for whole transcriptome amplification, reagents necessary for various biochemical reactions, such as reverse transcription, RT PCR, in vitro transcription, rolling circle amplification, bisulfate treatment, DNA extraction, RNA extraction, protein extraction, genome editing, permeabilization, and in situ sequencing, and combinations thereof. Examples of the lysis solutions include alkaline lysis reagents and proteinases.

Suitable biochemical reaction reagents are reagents for the preparation of libraries for massively parallel sequencing, including transposases, ligases, and fragmentases.

Before introduction of the biochemical reaction reagent, a barrier structure may be formed on the biological samples and a hydrogel may be covered thereon to minimize diffusion of the biochemical material. For example, agarose is introduced on the biological samples on which a barrier structure is formed, a hydrogel is formed by hardening the agarose, and the biochemical reaction reagent is introduced thereon. As another example, the biochemical reaction reagent may be introduced by soaking with a hydrogel and covering the hydrogel on the biological samples.

The biochemical reaction reagent may be used to introduce a reagent for a reaction of a biomaterial on a DNA microarray substrate, a massively parallel sequencing substrate or a flow cell for massively parallel sequencing.

The biochemical reaction reagent may be a reagent for decrosslinking the cured curable material to form a barrier structure. In the case where a barrier structure is covered on the areas where the target specimens are located, the biological samples around the areas where the barrier structure is present are removed by lysis, the decrosslinking reagent is supplied to expose the target specimens, followed by a biochemical reaction.

The biochemical reaction may include a reaction for ligation or insertion of reaction products in different microwells by injection of different types of oligonucleotides to tag the reaction products. The tagging may include injecting beads, microparticles, hydrogels, droplets or core shell particles attached with different types of oligonucleotides. The tagging may also include assembling a microarray substrate attached with oligonucleotides with a substrate on which biological samples are arranged. The biochemical reaction may also include ELISA, aptamer binding or a reaction for mass spectroscopy. Referring to the right diagram of FIG. 3, first, a predetermined amount of a lysis solution as the biochemical reaction reagent is dropped onto the target specimens. The lysis solution spreads to the areas where the target specimens are located and around the target specimens. As a result, only the target specimens present in the unmasked areas are lysed and the biological samples present in the masked areas are not lysed, enabling selective treatment of the target specimens without being contaminated by the other biological samples. The treated samples can be collected and analyzed in a separate space.

The masking structure formed on the substrate is patterned and the biochemical reaction reagent is introduced into the areas where the target specimens are located. The biochemical reaction reagent may be introduced in various ways.

In one embodiment, the masking structure may be formed mainly on the non-target specimens located in the vicinity of the target specimens when patterned for masking. At this time, the biochemical reaction reagent may be introduced into the areas where the target specimens are located and the masked areas located around the target specimens to prevent the target specimens from being contaminated by the other samples.

Referring to 3-1 and 4 of FIG. 2, the biochemical reaction reagent can be conveniently introduced into the areas where the target specimens are located and the masked areas around the target specimens. There is no particular restriction on the method for introducing the biochemical reaction reagent. The biochemical reaction reagent may be introduced by inkjet printing, microdispensing or large-capacity pipetting. That is, the biochemical reaction reagent may be conveniently introduced using general large-capacity pipetting means so long as it does not affect places distant from the target specimens. That is, since the surrounding non-target specimens are protected by masking, there is no need to deliberately introduce the biochemical reaction reagent into the limited areas where the target specimens are located by a precise technique such as inkjet printing to prevent contamination by the non-target specimens.

For example, although the areas of the target specimens have a size of several μm or less, the area treated by the biochemical reaction reagent may be in the range of tens of μm to several mm.

In a further embodiment, the masking structure may also be formed on a large area by coating a masking material over the entire surface of the substrate and patterning the masking material.

Referring to 3-2 and 4 of FIG. 2, the areas of the target specimens are exposed and the masking structure is formed around the target specimens over a broader area than the areas of the target specimens. In this case, the masking structure may form a single large-area film layer over the entire surface of the substrate. The masking film layer together with the non-target specimens may be peeled from the substrate when it has poor adhesion to the substrate but has a high bonding strength to the non-target specimens. As a result, the non-target specimens are completely removed and only the target specimens are left on the substrate.

In this embodiment, there is no particular restriction on the area that can be treated with the biochemical reaction reagent, thus being advantageous in that the treatment with the biochemical reaction reagent is freer than that in the previous embodiments. In addition, the target specimens are easy to recover and analyze in the subsequent step because other samples are not present on the substrate. In step 150, the target specimens are analyzed on the substrate or are recovered from the substrate and analyzed.

The target specimens reacted with the biochemical reaction reagent can be analyzed on the substrate. Alternatively, the target specimens may be recovered from the substrate and the recovered solution may be used for massively parallel next generation sequencing (NGS), mass spectrometry, and RNA-seq. The reaction solution of the target specimens can be recovered using a micromanipulator, a liquid handler, ultrasonic waves or micropipetting.

According to a further embodiment of the present disclosure, biological samples may be selectively analyzed by the following procedure. First, a substrate on which biological samples are arranged is prepared. Next, the substrate is divided into areas where one or more target specimens are located and areas where one or more non-target specimens are located. The division may include reading the kinds and locations of the biological samples.

Subsequently, a masking structure is formed to selectively mask the areas where the non-target specimens are located. A lysis solution is introduced into the areas where the target specimens are located to lyse the target specimens. Nucleic acid molecules originating from the target specimens by the lysis are treated with a biochemical reaction reagent to prepare libraries of the nucleic acid molecules for sequencing. Next, the libraries are recovered from the substrate. Subsequently, the recovered libraries are sequenced to selectively analyze the biological samples on the substrate.

Preferably, the sequencing may be performed by a high-throughput sequencing technique such as massively parallel next generation sequencing with very high analytical efficiency.

This sequencing can provide optical and electromagnetic signals together with positional information. The optical and electromagnetic signals are sequentially generated depending on the nucleotide sequence types. The use of massively parallel next generation sequencing enables simultaneous analysis of hundreds of thousands of sequences. Thus, massively parallel next generation sequencing can provide statistical data on the sequences of the analyte specimens with higher throughput than traditional sequencing techniques.

According to another embodiment of the present disclosure, the method for selective treatment of target specimens may include removing the masking structure together with the biological samples other than the target specimens. This step is easily carried out by a physical force without damage to the target specimens. The entire procedure of the method will be explained below.

First, a substrate on which biological samples are prepared. Next, the kinds and locations of the biological samples are read to divide the substrate into areas where one or more target specimens are located and areas where one or more non-target specimens are located. Subsequently, a masking structure is formed to selectively mask the areas where the non-target specimens are located. The masking structure together with the masked non-target specimens is peeled from the substrate to remove the non-target specimens, leaving the target specimens on the substrate. Subsequently, the target specimens are allowed to react with a biochemical reaction reagent and are then analyzed.

According to one embodiment, a biochemical reaction reagent is introduced into the areas where the target specimens are located, and the target specimens are allowed to react with the biochemical reaction reagent and are analyzed on the substrate. According to an alternative embodiment, the target specimens remaining unpeeled are recovered from the substrate by scraping with a suitable tool such as a knife and are analyzed by reaction with a biochemical reaction reagent. These embodiments associated with this selective treatment of biological samples are the same as those described in 3.2 and 4 of FIG. 2.

According to one embodiment, an adhesive or a material including a cell lysis reagent may be supplied to the areas where the target specimens are located, to physically separate the target specimens from the non-target specimens. Then, the target specimens are recovered from the substrate and are allowed to react with the biochemical reaction reagent for analysis.

The adhesive or the material including a cell lysis reagent may be in the form of a liquid, solid, polymer or hydrogel but is not limited thereto. The adhesive or the material including a cell lysis reagent may be in the form of particles with a diameter of 0.1 μm to 1 mm, preferably 1 μm to 100 μm.

Another embodiment of the present disclosure provides a method for selective treatment of target specimens using a microfluidic structure. The method includes i) preparing a substrate on which biological samples are arranged and ii) reading the kinds and locations of the biological samples to divide the substrate into areas where one ore more target specimens are located and areas where one ore more non-target specimens are located, as in the previous embodiments. Subsequently, iii) a first microfluidic structure is provided on the substrate. The first microfluidic structure may be a microfluidic chip or chamber. The first microfluidic structure may have at least one opening through which a fluid such as a curable material enters and exits. The first microfluidic structure may cover the substrate so as to surround the areas where the target specimens are located. Due to this structure, separate spaces are formed between the target specimens and the first microfluidic structure. The width between the substrate and the bottom of the ceiling of the first microfluidic structure in the separate spaces may be from 1 to 500 μm.

Next, iv) a curable material is introduced into the first microfluidic structure to fill the separate spaces. Then, v) energy is selectively applied to the curable material present in the areas where the non-target specimens are located, such that the curable material is cured to form a masking structure. The energy may be heat or light. Lithography may be used for the selective energy application. When the curable material is supplied based on microfluidics, the height of the masking structure may be limited depending on the size of the separate spaces. Microfluidics can ensure uniform supply of the curable material over the entire surface of the substrate, thus being advantageous in reducing the consumption of the curable material.

Next, vi) the first microfluidic structure is removed from the substrate. As a result, the masking structure is arranged in areas other than the areas of the target specimens on the substrate.

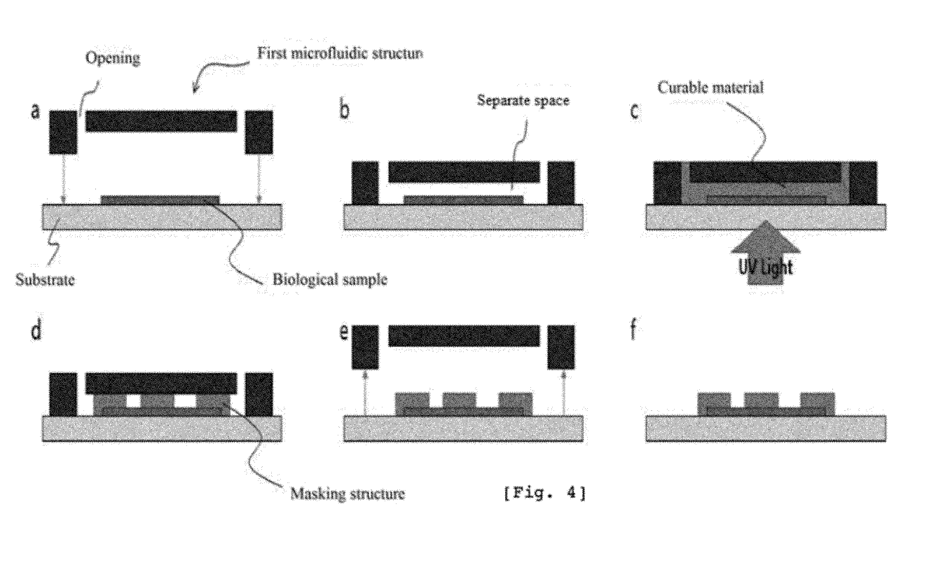

FIG. 4 illustrates the procedure for forming the masking structure using the microfluidic structure.

Next, vii) a biochemical reaction reagent is introduced into the areas where the target specimens are located, and is allowed to react with the target specimens. Finally, viii) the target specimens are analyzed on the substrate or are recovered from the substrate and analyzed. This procedure enables selective analysis of the biological samples.

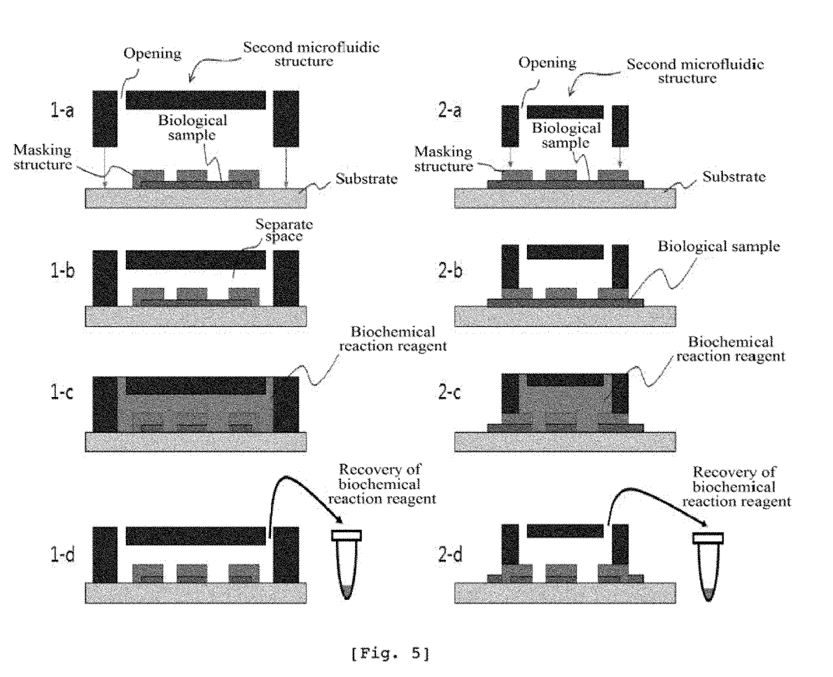

In one embodiment, steps vii) and viii) may be carried out based on microfluidics. FIG. 5 illustrates procedures for supplying and recovering the biochemical reaction reagent using the microfluidic structure. Referring to FIG. 5, the biochemical reaction reagent is supplied using the microfluidic structure and the masking structure is accommodated in the microfluidic structure (1-a to 1-d of FIG. 5).

Alternatively, the microfluidic structure may be designed to come into contact with the top of the masking structure. Due to this design, the biochemical reaction reagent may be selectively supplied to some of the unmasked areas depending on the size or structure of the microfluidic structure (2-a to 2-d of FIG. 5).

In one embodiment, another microfluidic structure may be used to introduce and recover the biochemical reaction reagent. Specifically, a second microfluidic structure is provided on the substrate on which the masking structure is formed. The second microfluidic structure may be a microfluidic chip or chamber. The second microfluidic structure may have at least one opening through which a fluid such as the biochemical reaction reagent enters and exits.

The second microfluidic structure may have the same structure as the first microfluidic structure. In this case, the first microfluidic structure present on the substrate may be used as the second microfluidic structure.

The second microfluidic structure may be arranged outside the periphery of the masking structure (1-b of FIG. 5) or in close contact with the top of the masking structure (2-b of FIG. 5). With this arrangement, separate spaces in which the areas of the target specimens are located may be formed to retain the biochemical reaction reagent. Referring to 1-b of FIG. 5, a chamber may be formed irrespective of the shape of the masking structure by a general approach. Alternatively, the biochemical reaction reagent may be selectively supplied to some of the unmasked areas depending on the size or structure of the microfluidic structure, as illustrated in 1-b of FIG. 5.

Next, the biochemical reaction reagent is introduced into the second microfluidic structure to react with the target specimens. After completion of the reaction, the target specimens are recovered from the second microfluidic structure and are analyzed.

In a further embodiment, the biochemical reaction reagent may be supplied in a state in which the first microfluidic structure is arranged, as illustrated in d of FIG. 4, without using the second microfluidic structure for introduction and recovery of the biochemical reaction reagent.

As described above, when the biochemical reaction reagent is supplied after assembly of the microfluidic structure on the substrate, the reagent is supplied to limited separate spaces. Thus, the consumption of the reagent can be reduced and the evaporation of the reagent can be prevented.

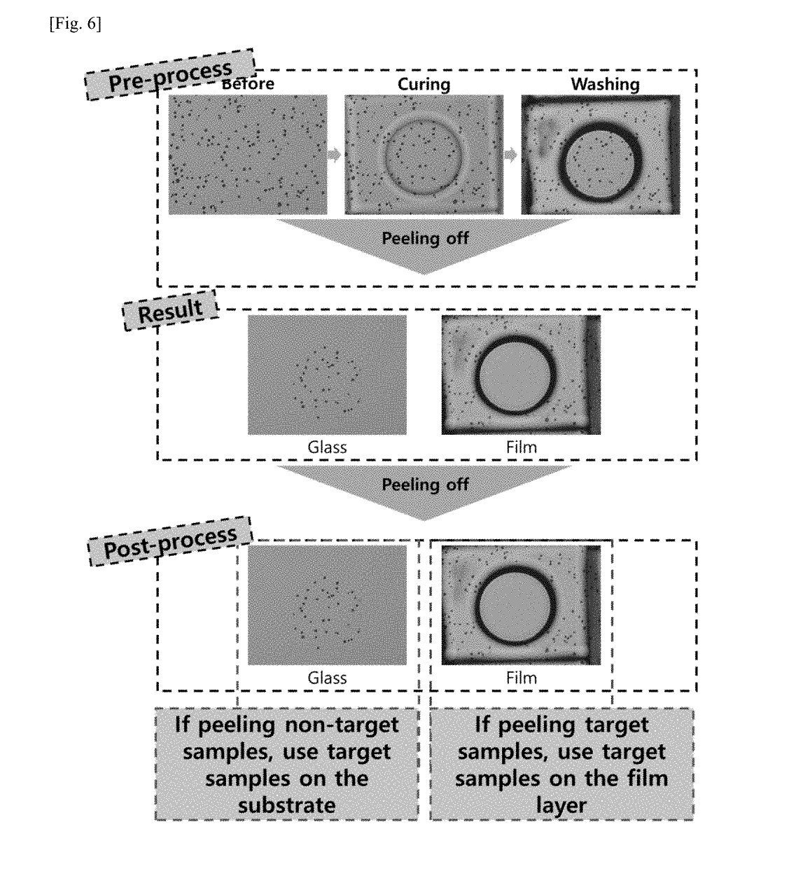

In a further embodiment of the present disclosure provides any positive selection of target samples by peeling film layer, as illustrated in FIG. 6. FIG. 6 indicates that the inventors can peel non-target samples in order to leave only target samples on the substrate or that the inventors can selectively fix target samples on the film layer. Specifically, the present disclosure provides a method for selective analysis of biological samples, comprising the steps of: preparing a substrate on which biological samples are arranged; dividing the substrate into areas where one or more target specimens are located and areas where one or more non-target specimens are located; forming a masking film layer on the substrate to selectively mask the areas where the target specimens are located; peeling the masking film layer from the substrate to remove the target specimens, leaving the target specimens on the film layer; introducing a biochemical reaction reagent into the film layer where the target specimens are located, such that the biochemical reaction reagent reacts with the target specimens or recovering the target specimens from the film layer and reacting a biochemical reaction reagent with the recovered target specimens; and analyzing the reacted target specimens.

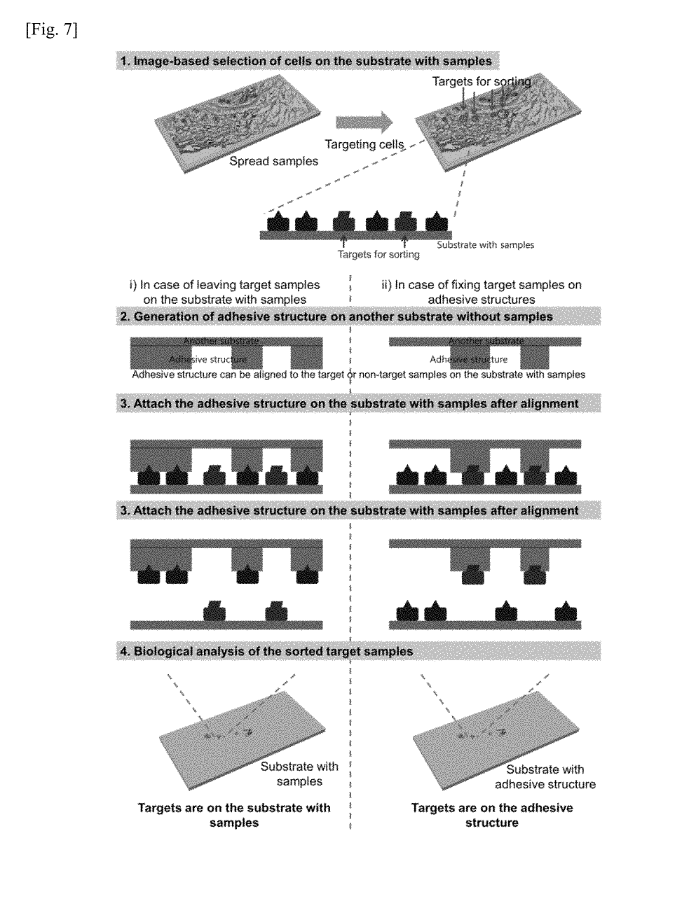

In a further embodiment of the present disclosure provides types such as using additional adhesive structure to peel off non-targets, or using additional adhesive structure to select target samples by peeling adhesive structure, as illustrated in FIG. 7. FIG. 7 indicates that the inventors don't have to generate film layer on the substrate with samples. The inventors can prepare another substrate to have adhesive structures which can be fit to the target or non-target samples after alignment. Then, the inventors can align and attach the substrate with samples and the substrate with adhesive structures. The inventors then can perform biological assay to the sorted target samples.

Specifically, the present disclosure provides a method for selective analysis of biological samples, comprising the steps of: preparing a substrate on which biological samples are arranged; dividing the substrate into areas where one or more target specimens are located and areas where one or more non-target specimens are located; preparing another substrate without biological samples; forming adhesive structures selectively on the substrate without biological samples, to be contact only to the one or more non-target specimens if the structures are aligned with the substrate with biological samples; aligning and contacting adhesive structures on the substrate without biological samples with substrates with biological samples; peeling the adhesive structures from the substrate with biological samples to remove the non-target specimens, leaving the target specimens on the substrate; introducing a biochemical reaction reagent into the substrate where the target specimens are located, such that the biochemical reaction reagent reacts with the target specimens or recovering the target specimens from the substrate and reacting a biochemical reaction reagent with the recovered target specimens; and analyzing the reacted target specimens.

Specifically, the present disclosure provides a method for selective analysis of biological samples, comprising the steps of: preparing a substrate on which biological samples are arranged; dividing the substrate into areas where one or more target specimens are located and areas where one or more non-target specimens are located; preparing another substrate without biological samples; forming adhesive structures selectively on the substrate without biological samples, to be contact only to the one or more target specimens if the structures are aligned with the substrate with biological samples; aligning and contacting adhesive structures on the substrate without biological samples with substrates with biological samples; peeling the adhesive structures from the substrate with biological samples to remove the target specimens, leaving the target specimens on the adhesive structures; introducing a biochemical reaction reagent into the adhesive structures where the target specimens are located, such that the biochemical reaction reagent reacts with the target specimens or recovering the target specimens from the adhesive structures and reacting a biochemical reaction reagent with the recovered target specimens; and analyzing the reacted target specimens.

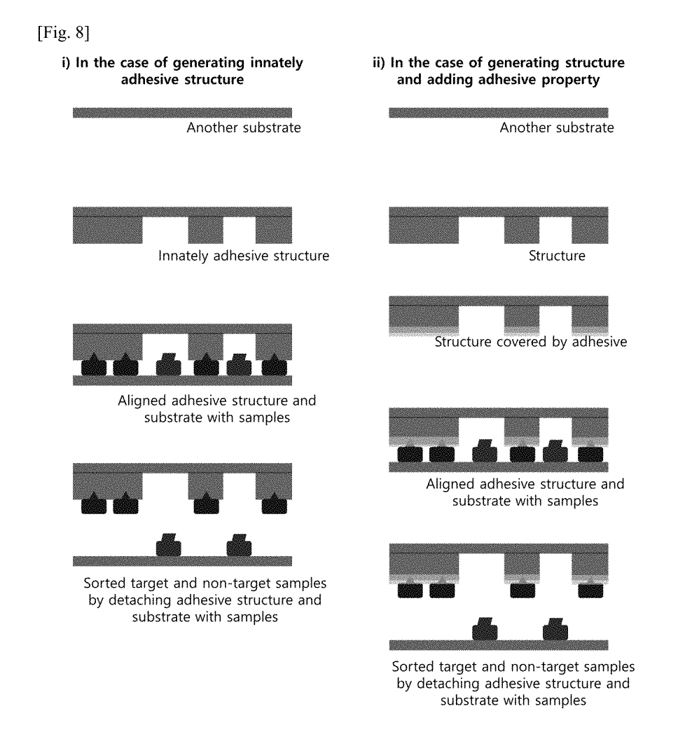

In a further embodiment of the present disclosure provides how can the inventors generate innately adhesive structures or how the inventors generate adhesive structures (additional step of gluing), as illustrated in FIG. 8. FIG. 8 indicates that the inventors can prepare another substrate with adhesive structure in many ways. The inventors can fabricate innately adhesive structure on another structure or can cover adhesive after fabricating structure on another substrate.

Specifically, the adhesive structure is formed by a technique selected from the group consisting of lithography, inkjet printing, and 3D printing at the same time having innately adhesive property, or the adhesive structure is formed by sequential lithography on several areas of the curable material using a lens between a mask and the substrate and additional step by covering or applying adhesive on the structure which is formed by sequential lithography.

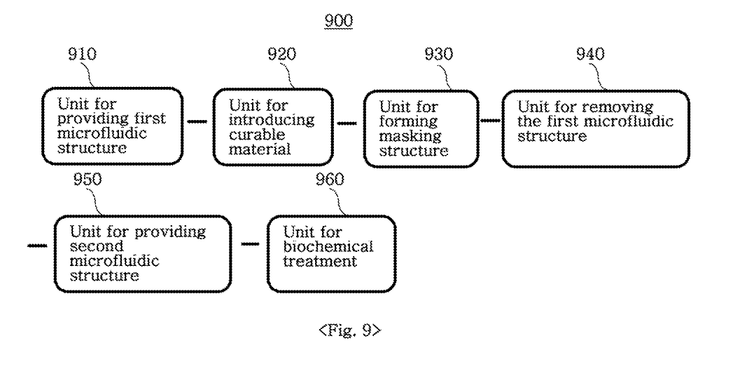

One embodiment of the present disclosure provides an apparatus for selective treatment of biological samples. FIG. 9 illustrates an apparatus for selective treatment of biological samples according to one embodiment of the present disclosure. Referring to FIG. 9, the apparatus 900 may include i) a unit 910 for providing a first microfluidic structure forming a masking structure on a substrate on which biological samples are arranged, ii) a unit 920 for introducing a curable material into the first microfluidic structure, iii) a unit 930 for forming a masking structure by applying energy to masking areas as per a user's request or a predetermined algorithm to cure the masking areas, iv) a unit 940 for removing the first microfluidic structure from the substrate, v) a unit 950 for providing a second microfluidic structure adapted to retain a biochemical reaction reagent on the substrate, and vi) a unit 960 for biochemical treatment by applying energy of light, heat, agitation, vibration or sound waves such that a biochemical reaction takes place.

Each of the units 910 and 950 may include an electrically driven stage, a motor, and an actuator.

The unit 930 may include a lithography system, an inkjet printing system or a 3D printing system. For example, the unit 930 may include an optofluidic maskless lithography system. To this end, the unit 930 may include a UV light source, a digital mirror device, and a lens.

The unit 960 may include means for storing the biochemical reaction reagent and means for supplying the biochemical reaction reagent. In one embodiment, the unit 960 may include a reaction promoting device for applying a physical force such as energy agitation, vibration or ultrasonic waves to the reaction spaces where the target specimens are located. In one embodiment, the unit 960 may further include a temperature controller for controlling the reaction temperature.

The apparatus may include some or all of the above-described elements. In the case where the first microfluidic structure is used to introduce the biochemical reaction reagent, the need to use the second microfluidic structure is eliminated, and as a result, the units 940 and 950 are omitted. When the first microfluidic structure is artificially formed, the unit 910 may be optionally omitted.

The use of the apparatus enables accurate and selective treatment of target specimens from biological samples including target specimens in an economical and rapid manner. Therefore, the apparatus can be used for subsequent selective analysis of biological samples.

According to the methods for selective treatment or analysis of biological samples, an accurate determination can be made as to whether tissues (particularly, cancer tissues) have certain mutations or abnormalities.

For example, cancer tissues extracted from cancer patients may be sequenced by the following procedure. First, the cancer tissues are spread on slide glass and stained with a well-known staining reagent (e.g., Giemsa). Then, the desired cells are selectively treated and recovered under observation with a microscope. Finally, the recovered cells are sequenced.

That is, the present disclosure enables the separation of biological samples with high specificity based on their kinds and locations. In addition, the present disclosure enables the analysis of target specimens while maintaining their original structure and morphology in areas where the target specimens are located because the structures are prepared while maintaining their coated state.

Furthermore, according to the present disclosure, there is no need to transfer target specimens to a corresponding container or substrate for a subsequent reaction, reducing the probability of contamination and ensuring high accuracy. Particularly, existing biochemical analysis methods can be applied without involving complicated processes after treatment of the samples. Therefore, the present disclosure can be applied to selective cell analysis, protein analysis, and gene analysis. Based on these analyses, the present disclosure can also be applied to more advanced follow-up studies such as disease diagnosis and translational medicine.

Although the present disclosure has been described herein with reference to the foregoing embodiments, those skilled in the art will appreciate that various modifications are possible, without departing from the spirit and scope of the present disclosure.

1. A method for selective analysis of biological samples, comprising the steps of: preparing a substrate on which biological samples are arranged; dividing the substrate into areas where one or more target specimens are located and areas where one or more non-target specimens are located; forming a masking structure to selectively mask the areas where the non-target specimens are located; introducing a biochemical reaction reagent into the areas where the target specimens are located, such that the biochemical reaction reagent reacts with the target specimens; and analyzing the reacted target specimens on the substrate or recovering the reacted target specimens from the substrate and analyzing the recovered target specimens.

2. The method according to claim 1, wherein the kinds and locations of the biological samples are read by image observation, fluorescence signals or coordinate information.

3. The method according to claim 1, wherein the biological samples are selected from the group consisting of tissues, blood, cells, DNAs, RNAs, proteins, exosomes, metabolites, biopsy specimens, and mixtures thereof.

4. The method according to claim 1, wherein the masking structure is formed by a technique selected from the group consisting of lithography, inkjet printing, and 3D printing.

5. The method according to claim 1, wherein the formation of the masking structure comprises coating a curable material on the substrate and curing the curable material by light or heat.

6. The method according to claim 1, wherein the masking structure is formed by sequential lithography on several areas of the curable material using a lens between a mask and the substrate.

7. The method according to claim 1, wherein the biochemical reaction reagent is treated in the areas where the target specimens are located and the masked areas located around the target specimens to prevent the target specimens from being contaminated by the biological samples present in the unmasked areas.

8. The method according to claim 1, wherein the biochemical reaction reagent is selected from the group consisting of lysis solutions, PCR reagents, reagents for whole genome amplification, reagents for whole transcriptome amplification, and combinations thereof.

9. A method for selective analysis of biological samples, comprising the steps of: preparing a substrate on which biological samples are arranged; dividing the substrate into areas where one or more target specimens are located and areas where one or more non-target specimens are located; forming a masking film layer on the substrate to selectively mask the areas where the non-target specimens are located; peeling the masking film layer from the substrate to remove the non-target specimens, leaving the target specimens on the substrate; introducing a biochemical reaction reagent into the areas where the target specimens are located, such that the biochemical reaction reagent reacts with the target specimens or recovering the target specimens from the substrate and reacting a biochemical reaction reagent with the recovered target specimens; and analyzing the reacted target specimens.

10. A method for selective analysis of biological samples, comprising the steps of: preparing a substrate on which biological samples are arranged; dividing the substrate into areas where one or more target specimens are located and areas where one or more non-target specimens are located; forming a masking structure to selectively mask the areas where the non-target specimens are located; introducing a lysis solution into the areas where the target specimens are located, to lyse the target specimens; reacting nucleic acid molecules originating from the target specimens by the lysis with a biochemical reaction reagent to prepare libraries of the nucleic acid molecules for sequencing; recovering the libraries from the substrate; and sequencing the recovered libraries.

11. A method for selective treatment of biological samples, comprising the steps of: preparing a substrate on which biological samples are arranged; dividing the substrate into areas where one or more target specimens are located and areas where one or more non-target specimens are located; forming a masking structure to selectively mask the areas where the non-target specimens are located; and introducing a biochemical reaction reagent into the areas where the target specimens are located, such that the biochemical reaction reagent reacts with the target specimens.

12. The method according to claim 11, wherein the reaction reagent is treated in the areas where the target specimens are located and the masked areas located around the target specimens to prevent the target specimens from being contaminated by the biological samples present in the unmasked areas.

13. A method for selective treatment of biological samples, comprising the steps of: preparing a substrate on which biological samples are arranged; dividing the substrate into areas where one or more target specimens are located and areas where one or more non-target specimens are located; forming a masking structure to selectively mask the areas where the non-target specimens are located; peeling the masking structure together with the masked non-target specimens from the substrate to remove the non-target specimens, leaving the target specimens on the substrate; and introducing a biochemical reaction reagent into the areas where the target specimens are located or recovering the target specimens from the substrate and reacting a biochemical reaction reagent with the recovered target specimens.

14. A method for selective treatment of biological samples, comprising the steps of: (a) preparing a substrate on which biological samples are arranged; (b) dividing the substrate into areas where one or more target specimens are located and areas where one or more non-target specimens are located; (c) providing a first microfluidic structure on the substrate; (d) introducing a curable material into the first microfluidic structure; (e) selectively applying energy to the curable material present in the areas where the non-target specimens are located, such that the curable material is cured to form a masking structure; (f) introducing a biochemical reaction reagent into the areas where the target specimens are located, such that the biochemical reaction reagent reacts with the target specimens; and (g) analyzing the reacted target specimens on the substrate or recovering the reacted target specimens from the substrate and analyzing the recovered target specimens.

15. The method according to claim 14, further comprising the step of removing the first microfluidic structure from the substrate between steps (e) and (f).

16. The method according to claim 14, wherein the first microfluidic structure covers the substrate so as to surround the areas where the target specimens are located to form separate spaces between the target specimens and the first microfluidic structure.

17. The method according to claim 14, wherein the step of introduction of a biochemical reaction reagent for a reaction with the target specimens and the step of recovery and analysis of the target specimens are carried out based on microfluidics.

18. The method according to claim 14, wherein step (f) further comprises: f-1) removing the first microfluidic structure from the substrate; f-2) providing a second microfluidic structure on the substrate to form separate spaces between the second microfluidic structure and the substrate; and f-3) introducing a biochemical reaction reagent into the second microfluidic structure to react with the target specimens.

19. An apparatus for selective treatment of biological samples, comprising: a unit for providing a first microfluidic structure forming a masking structure on a substrate on which biological samples are arranged; a unit for introducing a curable material into the first microfluidic structure; a unit for forming a masking structure by applying energy to masking areas as per a user's request or a predetermined algorithm to cure the masking areas; a unit for removing the first microfluidic structure from the substrate; a unit for providing a second microfluidic structure adapted to retain a biochemical reaction reagent on the substrate; and a unit for biochemical treatment by introducing a biochemical reaction reagent into separate spaces between the second microfluidic structure and the substrate or applying energy of light, heat, agitation, vibration or sound waves to the separate spaces such that a biochemical reaction takes place.

20. The apparatus according to claim 19, wherein the unit for forming a masking structure comprises a lithography system, a laser scanning system, an inkjet printing system or a 3D printing system.

21. The apparatus according to claim 19, wherein the unit for biochemical treatment comprises a storage element for storing the biochemical reaction reagent and a supply element for supplying the biochemical reaction reagent.

22. An apparatus for selective treatment of biological samples, comprising: a unit for providing a first microfluidic structure forming a masking structure on a substrate on which biological samples are arranged; a unit for introducing a curable material into the first microfluidic structure; a unit for forming a masking structure by applying energy to masking areas as per a user's request or a predetermined algorithm to cure the masking areas; and a unit for biochemical treatment by introducing a biochemical reaction reagent into the first microfluidic structure or applying energy of light, heat, agitation, vibration or sound waves to the first microfluidic structure such that a biochemical reaction takes place.

23. An apparatus for selective treatment of biological samples, comprising: a unit for introducing a curable material into a first microfluidic structure; a unit for forming a masking structure by applying energy to masking areas as per a user's request or a predetermined algorithm to cure the masking areas; and a unit for biochemical treatment by introducing a biochemical reaction reagent into the first microfluidic structure or applying energy of light, heat, agitation, vibration or sound waves to the first microfluidic structure such that a biochemical reaction takes place, wherein the first microfluidic structure is provided on a substrate on which biological samples are arranged.

24. A method for selective analysis of biological samples, comprising the steps of: preparing a substrate on which biological samples are arranged; dividing the substrate into areas where one or more target specimens are located and areas where one or more non-target specimens are located; forming a masking film layer on the substrate to selectively mask the areas where the target specimens are located; peeling the masking film layer from the substrate to remove the target specimens, leaving the target specimens on the film layer; introducing a biochemical reaction reagent into the film layer where the target specimens are located, such that the biochemical reaction reagent reacts with the target specimens or recovering the target specimens from the film layer and reacting a biochemical reaction reagent with the recovered target specimens; and analyzing the reacted target specimens.

25. A method for selective analysis of biological samples, comprising the steps of: preparing a substrate on which biological samples are arranged; dividing the substrate into areas where one or more target specimens are located and areas where one or more non-target specimens are located; preparing another substrate without biological samples; forming adhesive structures selectively on the substrate without biological samples, to be contact only to the one or more non-target specimens if the structures are aligned with the substrate with biological samples; aligning and contacting adhesive structures on the substrate without biological samples with substrates with biological samples; peeling the adhesive structures from the substrate with biological samples to remove the non-target specimens, leaving the target specimens on the substrate; introducing a biochemical reaction reagent into the substrate where the target specimens are located, such that the biochemical reaction reagent reacts with the target specimens or recovering the target specimens from the substrate and reacting a biochemical reaction reagent with the recovered target specimens; and analyzing the reacted target specimens.

26. A method for selective analysis of biological samples, comprising the steps of: preparing a substrate on which biological samples are arranged; dividing the substrate into areas where one or more target specimens are located and areas where one or more non-target specimens are located; preparing another substrate without biological samples; forming adhesive structures selectively on the substrate without biological samples, to be contact only to the one or more target specimens if the structures are aligned with the substrate with biological samples; aligning and contacting adhesive structures on the substrate without biological samples with substrates with biological samples; peeling the adhesive structures from the substrate with biological samples to remove the target specimens, leaving the target specimens on the adhesive structures; introducing a biochemical reaction reagent into the adhesive structures where the target specimens are located, such that the biochemical reaction reagent reacts with the target specimens or recovering the target specimens from the adhesive structures and reacting a biochemical reaction reagent with the recovered target specimens; and analyzing the reacted target specimens.

27. The method according to claim 25, wherein the adhesive structure is formed by a technique selected from the group consisting of lithography, inkjet printing, and 3D printing at the same time having innately adhesive property.

28. The method according to claim 25, wherein the adhesive structure is formed by sequential lithography on several areas of the curable material using a lens between a mask and the substrate and additional step by covering or applying adhesive on the structure which is formed by sequential lithography.

29. The method according to claim 26, wherein the adhesive structure is formed by a technique selected from the group consisting of lithography, inkjet printing, and 3D printing at the same time having innately adhesive property.

30. The method according to claim 26, wherein the adhesive structure is formed by sequential lithography on several areas of the curable material using a lens between a mask and the substrate and additional step by covering or applying adhesive on the structure which is formed by sequential lithography.
